(19) United States
(12) Patent Application Publication
(10) Pub. No.: US 2008/0266840 A1
(43) Pub. Date: Oct. 30, 2008

(54) SURGICAL ILLUMINATION DEVICE

(75) Inventors: Michael Nordmeyer, Pittstown, NJ (US); Eric David Serafin, Phillipsburg, NJ (US); Glenn William Ruhf, Branchburg, NJ (US); Raymond Louis Nordmeyer, Nazareth, PA (US)

Correspondence Address: SYNNESTVEDT & LECHNER, LLP, 1101 MARKET STREET, SUITE 2600, PHILADELPHIA, PA, 19107-2950, US

(73) Assignee: ENGINEERED MEDICAL SOLUTIONS COMPANY, LLC, Phillipsburg, NJ (US)

(21) Appl. No.: 12/029,829

(22) Filed: Feb. 12, 2008

Related U.S. Application Data
(60) Provisional application No. 60/889,468, filed on Feb. 12, 2007.
(60) Provisional application No. 60/950,313, filed on Jul. 17, 2007.

(51) Int. Cl. A61B 1/06

(52) U.S. Cl. 362/119

(57) ABSTRACT

A surgical illumination device is disclosed. The device has a light source mounted at the end of a flexible boom. The boom is attached to a housing containing a battery and an electronic controller. A switch actuator on the housing allows a user to turn the device on and off through the controller.

### CROSS REFERENCE TO RELATED APPLICATIONS

This application claims priority to U.S. Provisional Application Nos. 60/889468, filed Feb. 12, 2007, and 60/950313 filed Jul. 17, 2007, both of which are hereby incorporated herein by reference.

### FIELD OF THE INVENTION

This invention relates to illumination devices suitable for use during medical examinations and surgical procedures.

### BACKGROUND

Ambient illumination is often not adequate for medical examinations and surgical procedures. Regardless of the intensity of the ambient lighting, shadows, for example, cast by the physician or by draperies during a gynecological examination may prevent proper illumination of the examination site. Surgical procedures within body cavities may also require a source of illumination which can be positioned within the cavity to properly illuminate its interior, as there is usually no direct line of sight between the ambient source of illumination and the cavity interior.

Illumination devices useful in such environments must also be reliable, capable of being sterilized, capable of operating with other surgical instruments and should be easy for a physician to manipulate with one hand while wearing gloves. There is clearly a need for illumination devices which can provide light that adequately illuminates an examination site or the site of a surgical procedure.

Concerns present with such illumination devices include brightness of the light, temperature of the light, battery life, shelf life of the device, ease of use with other devices and affordability, among others. For example, a clear bright light of suitable intensity may generate sufficient heat to cause tissue damage to the patient or to members of the medical team.

As another example, the illumination devices should preferably be useable immediately upon removal from a hermetically sealed packaging. This requires the illumination device to be packaged and shipped to the medical user ready for use, e.g., the illumination device was shipped to the user fully assembled with the battery installed. Nevertheless, batteries have a limited shelf life, particularly when installed in a working device. Thus it is desirable to have an illumination device with an improved shelf life and which will last as long as possible once use begins.

It is also desirable to have an illumination device that is as user friendly as possible, and capable of being quickly moved and mounted and removed and remounted as needed for the medical procedure being carried out.

Known illumination devices include those disclosed in U.S. Pat. No. 6,428,180 to Karram et al and U.S. Pat. No. 7,270,439 to Horrell et al., both of which are incorporated by reference herein. Nevertheless, further improvements over these devices are believed to be advantageous to address various concerns, including those discussed above.

### SUMMARY

The invention concerns an illumination device comprising an electrically powered light source. In one form, the light source is mounted on one end of a flexible boom. A housing is attached to the opposite end of the boom. An electric battery is located within the housing, the battery being operatively associated with the light source for providing power thereto. A switch actuator is mounted on the housing for controlling flow of electricity from the battery to the light source to turn the illumination device on and off.

In one embodiment the light source comprises a light emitting diode. A lens may be mounted on the boom overlying the light source to focus the light emitted from the light source. The device may further comprise an electronic controller positioned within the housing. The controller is actuated by the switch actuator that communicates with the controller via a switch such as a push button, signaling the controller that the switch has been activated. If the light is off when the switch is activated, the controller will turn on the light. When the light is on when the switch is activated, the controller will turn off the light.

The illumination device can also include at least one cradle shaped to receive and support a portion of the illumination device, such that the illumination device is releasably attachable to said cradle. Various forms of such cradles provide flexibility in use of the device, e.g., a cradle can be used to attach the illumination device to an instrument, or to mount the illumination device to a surface. The illumination device can be moved back and forth between the various cradles, or even between multiple cradles of the same type, e.g., between two surface mounted cradles as may be needed during the procedure being carried out.

The invention also provides for a retractor and/or suction device attachable with the illumination device.

The invention also encompasses a kit. The kit can comprise the illumination device, and/or a retractor, and/or a suction device, and/or one or more cradles, and/or any other combination of tools, instruments and devices.

### BRIEF DESCRIPTION OF THE DRAWINGS

FIG. 1 is a perspective view of an exemplary illumination device according to the invention;

FIG. 2 is an exploded perspective view of the illumination device shown in FIG. 1;

FIG. 2A is an exploded view of a portion of the device shown in FIG. 2 on an enlarged scale;

FIG. 3 is a cross sectional view taken at line 3-3 of FIG. 1;

FIG. 4 is a cross sectional view taken at line 4-4 of FIG. 1;

FIG. 4A is a cross sectional view taken at line 4A of FIG. 4;

FIG. 5 is an exploded view of a kit and a package according to the invention;

FIG. 6 is a perspective view of an illumination device combined with a retractor having suction according to the invention;

FIG. 7 is a perspective view of an illumination device and one type of mounting cradle;

FIG. 7A is a enlarged top perspective view of a single retaining element shown in FIG. 7;

FIG. 8 is a detailed perspective view of a base portion of another type of a mounting cradle shown on an enlarged scale; and

FIG. 9 is a cross sectional view of an arming pin formed as a shear tab in the container bottom and which is used as an electrical interrupt.

### DETAILED DESCRIPTION

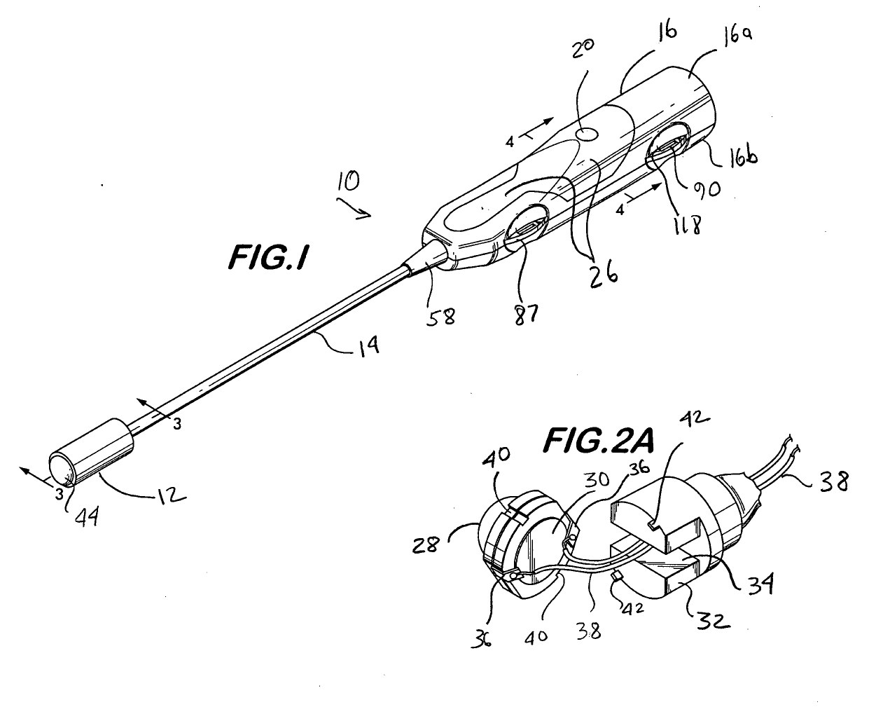

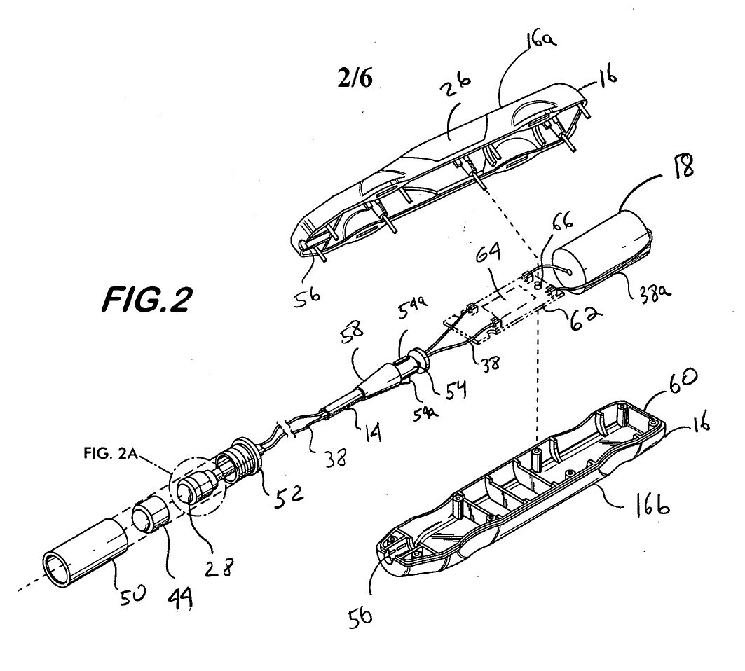

FIGS. 1 and 2 show an embodiment of an illumination device 10 according to the invention. Device 10 comprises an electrically powered light source 12 mounted on the end of a flexible boom 14. A housing 16 is mounted on the opposite end of the boom 14. The housing 16 holds an electrical battery 18 and has a switch actuator 20 for turning the device on and off by supplying electrical power from the battery to the light source 12.

The housing 16 can be made of any suitable material for medical use, and in the illustrated embodiment is made of an acrylic material such as Cyro G20 Hiflow™ formed in an injection molded process in two sections 16a, 16b as seen in FIG. 2. With further reference to FIGS. 4 and 4A, the switch actuator 20 is formed in an opening 22 in housing 16 of the same material and preferably as part of the molding process for the housing 16, suspended within the opening by two attachment members 24 having a flexure bend as shown to allow the switch actuator 20 to be depressed relative to the housing 16. As best seen in FIG. 2, the housing sections include internal press-fit interference connection pins, ribs, and other support members as known in the art to provide support for the various components to be mounted within, and to provide for an interference press-fit connection of the two housing sections. Some adhesive can be provided to at least the front two pins (LED side of housing 16) to prevent the housing halves 16a, 16b from separating when the boom 14 is flexed.

A skin layer 26 of a resilient material such as GLS Versaflex OM 1040X™ is molded over a top section of the housing 16 and in the opening 22 around the switch actuator 20 (see also FIG. 4). The skin layer 26 is flexible and resilient to allow depressing of the switch 20 and helps return the switch actuator to its initial position while supporting and maintaining a sealed connection thereto, and which provides a comfortable easy to grip feel (the opening 22 is filled in and covered by the over mold skin layer 26). A skin layer 26 of similar material can be provided along a section of the underside of the lower housing section 16b to add an easy to grip feel there as well (FIG. 4).

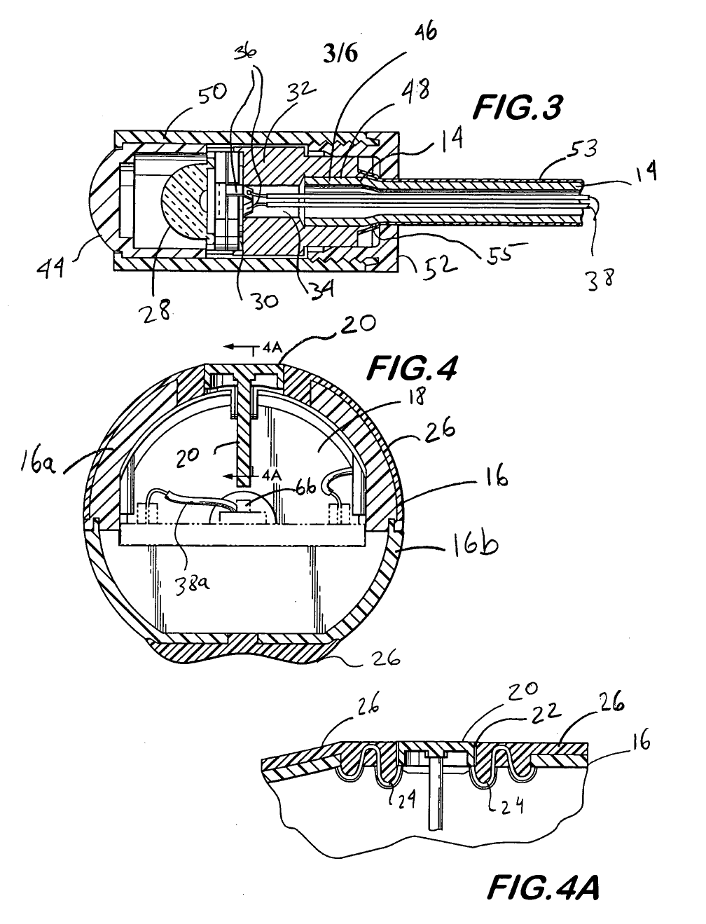

In the exemplary embodiment 10, the light source 12 comprises a light emitting diode (LED) 28, best shown in FIGS. 2A and 3. A preferred LED is provided by Luxeon, part #LXHL-PW09. Diode 28 has a metal base 30 which is in contact with a metal heat sink 32 (e.g., aluminum) mounted on the end of boom 14 to conduct heat away and prevent overheating of the LED during operation. The heat sink 32 has a channel 34 which receives contacts 36 on the diode. The contacts 36 are connected to conductors 38 which supply electrical energy to the diode from the battery 18 when the switch actuator 20 is used to turn on the device 10. Proper orientation of the diode 28 ensuring that the contacts are positioned within the channel and not in contact with the heart sink is provided by notches 40 on the diode which receive registration pins 42 extending from the heat sink 32. By maintaining proper orientation between the diode 28 and heat sink 32 with the registration pins 42, short circuiting of the diode is prevented.

As best shown in FIG. 3 a lens 44 is positioned overlying the light emitting diode 28. The lens is configured to focus or spread the light from the diode as desired. In the illustrated embodiment, the lens 44 focuses the light from the LED 28 to provide a concentrated beam. For example, in one preferred embodiment, the beam may encompass an angle up to 27° and concentrate the light over a 1.875 inch area (substantially square shape) at a distance of 4 inches from the lens. In another possible arrangement, the beam may encompass an angle up to 14° and concentrate the light over a 1.5 square inch area at a distance of 6 inches from the lens. Other suitable configurations may be used. In the illustrated embodiment, the lens 44 is formed of a clear plastic material such as Cyro G20 Hiflow™.

With reference to FIG. 3, the heat sink 32 preferably is attached to boom 14 by swaging the end of the boom, i.e., expanding the diameter of the boom at 46 to engage the inside surface of a bore 48 extending a set distance into the heat sink, thereby making a tight connection to fixedly hold the two members together and allow good heat conduction. The lens 44 and diode 28 are captured and held against the heat sink 32 by an elongated collar 50 that surrounds the lens and diode and engages a retaining base 52 positioned on the boom 14 adjacent to the heat sink. The collar 50 can be made of any suitable biocompatible material, such as a random copolymer polypropylene. Collar and base preferably interference snap fit together making a water tight connection, although other joining means are also feasible.

Boom 14 is preferably formed of a hollow aluminum tube that is readily bendable into a desired shape to allow the position and orientation of the light source 12 to be adjusted for convenient pointing when held in the hand, or relative to its target area when on a fixed mounting. The boom is yieldably adjustable so that it maintains the shape into which it is bent. In the illustrated embodiment, the boom is formed of annealed aluminum tubing having about a 0.125 inch outer diameter and about 25 thousandths of an inch wall thickness. The annealed aluminum is sufficiently soft to be malleable with minimal kinking or breakage. The boom 14 may be covered with a protective coating 53 as shown, for example a heat shrink material such as a medical grade high temperature fluoropolymer which protects the aluminum and affords advantage when the illumination device is sterilized. It is seen that that the protective coating 53 is sealed onto an end lip 55 of the heat sink 32. It is seen that the end lip 55 is tapered, creating an opening smaller than the outer diameter of the swaged section of the boom 14 and thereby helping to hold the boom tightly to the heat sink 32.

As best shown in FIG. 2, the housing 16 is mounted on the opposite end of the boom 14 from the light source 12 and provides a convenient handle for manual manipulation of the device. The end of the boom opposite the LED includes a connection collar 54, having tabs 54a, that is held within the receiving recess 56, having tab receiving openings (not shown), configured to cooperate with the collar 54. The collar 54 can be made of any suitable material, such as an acrylic material such as Cyro G20 Hiflow™. A boot 58, preferably made of a resilient material such as GLS Versaflex OM 1040X™, is provided on the end of the boom 14 where the boom meets the housing 16 as shown to prevent the boom from excessive bending at that position which could break the boom. The housing 16 may include a slot 60 that does not extend completely through the housing wall, but which is shaped to receive a tool, such as non sharp end of a scalpel, to separate the two housing halves 16a 16b from one another. This can be used for removal of the battery for recycling after use of the illumination device 10.

In addition to the battery 18, the housing 16 also contains a circuit board 62 with an electronic controller 64. The controller may comprise, for example, an integrated circuit which is programmed to control the operation of the illumination device as described below. As shown in FIG. 4, the switch actuator 20, mounted on the housing, interfaces with a switch 66 on the circuit board for manually turning the illumination device on and off. The switch 66 can be formed of any device and in any configuration capable of signaling the controller, such as the momentary pushbutton switch device as used in the preferred embodiment, and which is surface mounted on the circuit board. The preferred pushbutton is provided by Panasonic, part #EVQ-Q2F02W. In the illustrated embodiment, the switch actuator does not directly turn the illumination device on and off, but sends a signal to the controller via the micro switch 66, which controls the device according to its programming as described below. As indicated, wires 38 connect the LED to the controller, and wires 38a connect the battery to the controller. A CR123 lithium battery providing about 3V is electrically connected to the controller with wires in the illustrated embodiment.

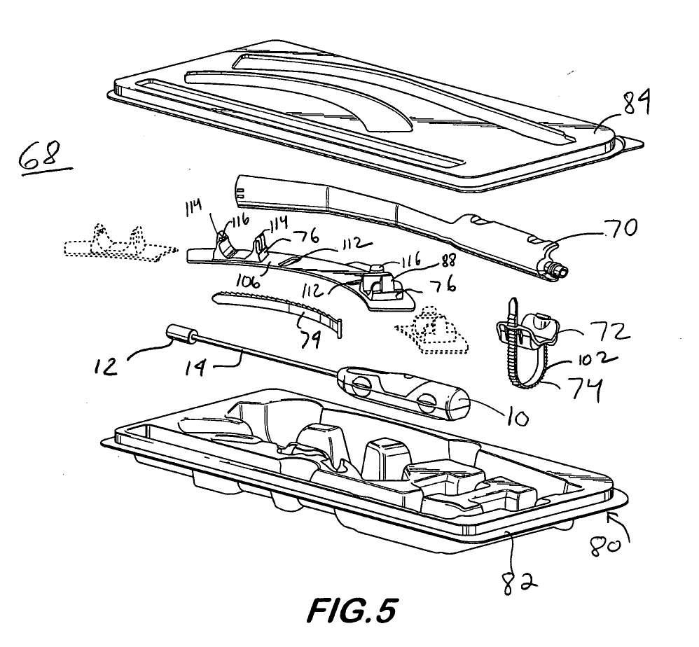

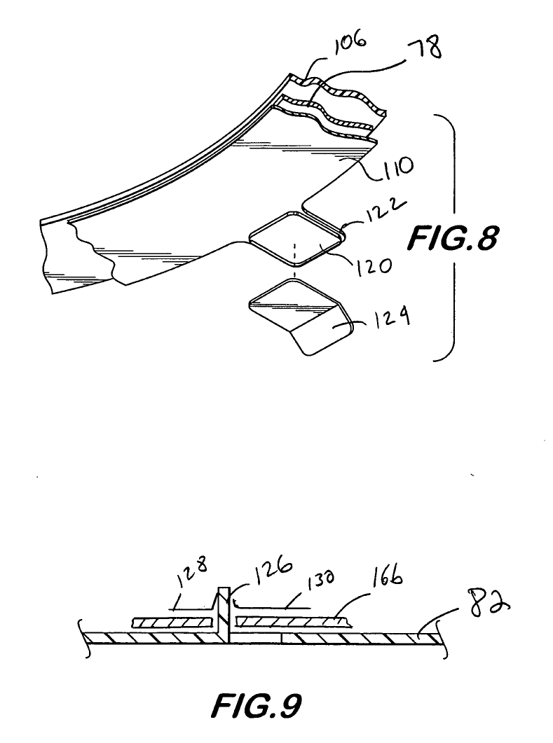

FIG. 5 illustrates a kit 68 encompassed by the invention. Kit 68 includes, along with the illumination device 10, one or more additional components 70 such as the instrument (retractor and suction device as shown), a mounting cradle 72 with a mounting strap 74, as well as a mounting cradle 76 having an adhesive layer 78 (see FIG. 8). The kit may be provided in a package 80 comprising a container 82 having a lid 84. The container and lid may be made of any suitable material such as Pentamed MD E678 Copolyester film material. The container and lid may be placed in a sterile enclosure, such as a TYVEK® pouch which allows a sterilizing gas to penetrate and then maintains the sterile environment within the pouch.

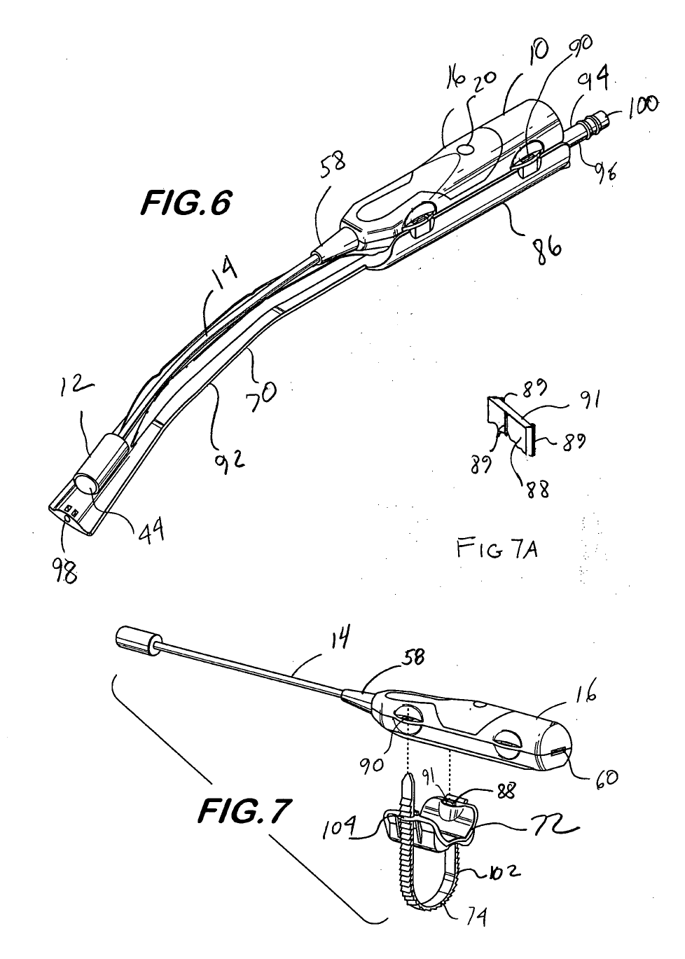

As shown in FIG. 6, in one form the instrument is a retractor 70 that has a base portion 86 which receives and retains the housing 16 for mounting the retractor onto the illumination device 10. The retractor 70 is preferably removably mounted and is retained by retaining elements 88, here formed of tabs that engage slots 90 integral with the housing 16 in a similar manner as does the retaining elements 88 of the cradle 72 further described below. The retractor 70 may be made from a plastic material such as Cyro G20 Hiflow™ which may be readily deformed to engage and disengage the housing and the base portion. A projecting portion 92 is attached to the base portion 86 and extends adjacent to the boom 14. In the illustrated embodiment, the instrument also forms a suction device 94 comprising a duct 96 that extends along and through the base and projecting portions of the retractor. The duct has an inlet 98 positioned at the end of the retractor projecting portion 92 and an outlet 100 positioned at the base portion 86. The outlet is connectable to a source of suction (not shown) during operation. The retractor need not have suction, and any suitable surgical tool can be modified to be attachable with the illumination device 10. This instrument 70 could also be a suction device by itself, without the retractor capability.

FIG. 7 illustrates in detail the mounting cradle 72 with a mounting strap 74. Cradle 72 is also formed of a suitable material such as Cyro G20 Hiflow™ and can include a skin layer on its bottom made of GLS Verseflex OM 1040X™. The cradle 72 receives the housing 16 therein and has retaining elements 88, such as longitudinal tabs as shown that extend upwardly to engage the longitudinal housing slots 90, permitting easy engagement and removal of the device 10 from the cradle 72. With further reference to FIG. 7A, it is seen that the retaining elements 88 for the cradle 72 include small ribs 89 formed on tabs 91 to provide a sufficient interference fit in the slot 90 to hold the housing 16 thereto. It is believed that there may be a slight deforming of the ribs and tab and/or the slot 90 to hold the two together. This is one possible configuration as any suitable arrangement or ribs, dots, or other shapes and forms for creating the interference fit can be used. In this example, the adjustable strap 74 has ratchet teeth 102 which engage a pawl 104 to mount the cradle on an item, such as another surgical instrument, allowing the illumination device to be used with the item without the need for another hand to hold the device 10.

FIGS. 5 and 8 illustrate another mounting cradle 76, which also is capable of receiving the housing 16 of the device 10, and further has a base 106 to which is attached the adhesive layer 78. The adhesive layer allows the cradle 76 to be attached to a surface, such as the draperies used during an examination or surgery. This permits the illumination device 10 to be positioned for maximum effectiveness during the examination or operation. The adhesive layer 78 is protected by a release sheet 110 which does not adhere strongly to the adhesive and is removed to expose the adhesive layer before use. As best seen in FIG. 5, each of the two mounting cradles 76 is detachable from the base 106 by cutting or breaking the area adjacent to openings 112 in the base 106. The base 106 in its entirety can be attached to a surface, such as a surgical drape, if additional adhesive surface is needed to support the illumination device 10 in one of the cradles 76. The openings 112 also allow the base 106 to be bent to match the contour of a surface as needed. Here the cradles 76 have retaining elements 88 formed of two resilient arms 114 having a retaining tab 116 disposed at the distal end of each arm 114. As the housing 16 of the illumination device 10 is pressed into the cradle 76, i.e., two opposing latch tabs 87 are pushed into the cradle 76, the resilient arms 114 move away from one another to allow the tabs 87 to pass, then returning to their original position whereby the retaining tabs 116 engage the top face 118 of the tabs 87 to hold the housing 16 in place. To remove the illumination device 10 from the cradle, a simple twisting of the housing 16 will move the arms 114 sufficiently to release the latches 116.

With reference to FIG. 8, a further advantage of the present invention is described. As discussed above, the base 106 includes a release sheet 110 that is removed to expose the adhesive layer 78 on the underside of the base 106. An adhesive layer 120 on sheet tab 122 can be provided on the underside of the release sheet 110 to adhere the release sheet to the bottom of the container 82. This allows removal of the base 106 from the container 82 and the automatic removal of the release liner 110 which stays adhered to the container, keeping the waste in the tray. A release sheet 124 on the adhesive layer 120 can be peeled to expose the adhesive when the base 106 is placed into the container 82 during assembly.

It is appreciated that the device 10 provides a universal quick connect means for quick attachment, detachment, and reattachment to another device as needed to change location or position of the illumination device 10. For example, the device 10 could be quickly docked to any cradle 72, 76, then adhered to a surgical drape, removed and re-docked to another cradle attached to a surgical instrument, attached to the retractor, etc.

In operation the switch actuator 20 is manually depressed to turn on the illumination device 10. The switch actuator actuates the switch 66 on controller 64. As discussed previously, the switch 66 of the present device is a pushbutton device. In the preferred embodiment, the switch actuator and switch 66 do not directly control the flow of electricity from the battery 18 to the light emitting diode 28. Instead, the switch 66 communicates with the controller by providing a signal to the controller which controls the flow of electrical energy according to the controller's programming. Pressing the switch actuator 20 turns the diode 28 on when it is off and off when it is on. Since the full current of the device does not flow through the switch 66, a smaller and less expensive switch can be used as compared to a switch through which the full electrical load of the illumination device would pass. Such switch devices, however, such as push buttons, may produce false signals immediately (microseconds) after being actuated due to “bouncing” of the contacts within the switch before the switch returns to a steady state. These false signals can be neutralized or filtered out in the illustrated embodiment with de-bounce programming, e.g., after a signal is received by the controller indicating that the state of the LED is to be changed (turned on or off), the controller is programmed to wait a predetermined number of program cycles to ensure that the switch 66 has returned to steady state before carrying out the desired action. In the present embodiment, the controller waits five program cycles before carrying out the desired action. If a change in state of the switch contacts is detected before the end of the five cycles, the counter starts over again to count five cycles. This continues until the program counts five cycles without detecting a change of state of the switch contacts. In practice this event happens in microseconds and prevents the bounces from operating the light. Without such de-bounce means, one could press the switch and not be sure which state the light would end up, on or off. An alternative means of de-bouncing would be to program the controller to ignore signals received from the switch 66 for a predetermined time after the switch was activated, e.g., the typical time it takes the switch contacts to return to a steady state.

When the LED 28 is turned on the controller is programmed to pulse current through the diode between high and low current levels. Pulsing the diode extends battery life and reduces the amount of heat produced. The LED is pulsed at a rate that is undetectable by the human eye so as to appear substantially continuous and bright. Here, The LED is electrically coupled to an LED driver circuit that outputs current to the LED at a predetermined level. The LED driver circuit is also electrically coupled to external resistors that are used to control the value of the current output from the driver circuit to the LED. By the controller switching the effective value of the external resistors from a first resistance to a second resistance, the current output from the driver circuit to the LED is varied from a predetermined high current level to a second predetermined lower current level at which the LED will have a lower brightness than when powered at the higher current level. The LED driver circuit is powered by a DC voltage source, for example a 3 Volt DC battery as discussed above. LED driver circuits such as this are known in the art. One example of an LED driver circuit is disclosed in a brochure published by Linear Technology for the LTC 3215 700 mA Low Noise High Current LED Charge Pump (LT 0306 REV A), which is hereby incorporated herein by reference.

The effective resistance of the external resistors is controlled according to a pulse train with a predetermined duty cycle. While the device is on, the pulse train continuously oscillates between a logic high level and a logic low level, with each logic high level lasting for about 36 microseconds and each logic low level lasting for about 50 microseconds. While the pulse train is at a logic high level, the effective resistance is set to the first resistance at which the LED has a lower brightness level. While the pulse train is at a logic low level, the effective resistance is set to the second resistance at which the LED has a higher brightness level. Therefore, as the pulse train oscillates from logic high to logic low, the brightness of the LED oscillates from a corresponding lower level to brighter level. Because the period of the pulse train is only 86 microseconds, the changes in the brightness of the LED are not discernable to the human eye. Thus, the LED appears to have a continuous level of brightness. Because the current output from driver circuit to the LED is reduced during each logic-high portion of each cycle of the pulse train, the power consumption of the device is thereby reduced.

The controller may also include a timer circuit. The timer works in conjunction with a battery interrupt feature programmed into the controller which permanently disables the illumination device 10 after a predetermined duration has elapsed (as measured by the timer) after a designated event has occurred. The time period of the predetermined duration may vary, but will be shorter than the battery life. This disabling feature prevents the illumination device from being used on more than one patient, and helps mitigate cross contamination and infection of patients.

In another embodiment illustrated in FIG. 9, the event which starts the timer is initially supplying electrical energy to the controller. This is effected by having a non-conducting arming pin 126 positioned between two electrical contacts 128 and 130. Contact 128 is in electrical communication with the battery 18. Contact 130 is in electrical communication with the controller 64. The arming pin keeps the contacts apart, preventing electrical energy from flowing to the controller. When the arming pin is removed, the contacts 128 and 130 engage one another and electrical energy is supplied to the controller. The timer begins to time the predetermined duration during which the illumination device 10 will be useable. At the end of the time period, the controller 64 will disable the device and prevent further use. In this example embodiment, the arming pin is part of the package 80 in which the kit 68 is provided, so removal of the illumination device from the package will start the timer which, along with the controller, determines the length of time which the illumination device will be operable.

It is understood that the above-identified arrangements are merely illustrative of the many possible specific embodiments which represent applications of the present invention. Numerous and varied other arrangements can readily be devised in accordance with the principles of the invention without departing from the spirit and scope of the invention.

What is claimed is:

1. An illumination device comprising:
an electrically powered light source;
a flexible boom having a first end on which said light source is mounted;
a housing attached to a second end of said boom opposite to said first end;
an electric battery located within said housing, said battery being operatively associated with said light source for providing power thereto;
a controller electrically connected to said battery and said light source for controlling the electrical power provided to said light source, and
a switch mounted on said device and in communication with said controller so as to provide a signal to said controller when said switch is actuated, said controller being actuated by said switch to control the electrical power to the light source for turning the light source on and off.

2. An illumination device according to claim 1, wherein said light source comprises a light emitting diode.

3. An illumination device according to claim 1, further comprising a lens mounted on said boom overlying said light source, said lens focusing light emitted from said light source.

4. An illumination device according to claim 1, further comprising means for pulsing the light source between high and low currents at a rate undetectable by the human eye so as to appear substantially continuous.

5. An illumination device according to claim 1 further comprising means for debouncing to minimize false signals from said switch.

6. An illumination device according to claim 1 further comprising at least one cradle shaped to receive and support said housing therein, said housing being releasably attachable to said cradle.

7. An illumination device according to claim 6 wherein said cradle includes a mounting strap attached thereto, said mounting strap being adjustable for attaching said illumination device to another item such as a medical instrument so as to be capable of attaching said illumination device to said another item.

8. An illumination device according to claim 6 wherein said cradle includes a base, said base having an adhesive thereon for attaching said base to a surface.

9. An illumination device according to claim 1, further comprising a retractor, said retractor having a base portion configured to be removably mountable on said housing and a projecting portion extending from said base portion adjacent to said boom.

10. An illumination device according to claim 1, further comprising a suction device, said suction device including a duct having an inlet and an outlet, said outlet being connectable to a source of suction for drawing fluid into said inlet from an area illuminated by said light source, said suction device having a base portion configured to be removably mountable on said housing and a projecting portion extending from said base portion adjacent to said boom.

11. An illumination device according to claim 1, further comprising a heat sink member fixedly attached to said first end of said boom, and in contact with said light source to conduct heat from said light source to said boom.

12. An illumination device according to claim 11, wherein one of said heat sink member and said light source includes registration pins, and the other of said heat sink member and said light source includes notches for receiving said registration pins so as to properly orient said heat sink member and said light source relative to one another.

13. An illumination device according to claim 1, further comprising a timer, wherein said controller is configured to permanently interrupt the flow of electricity to said light source thereby preventing further use of said illumination device after a predetermined time duration has elapsed.

14. An illumination device according to claim 6, wherein said cradle has retaining elements for cooperating with said housing to hold said housing to said cradle.

15. An illumination device according to claim 14 wherein said housing includes a slot, and said retaining elements include a tab configured to interference fit into said slot for holding said housing to said cradle.

16. An illumination device according to claim 1 in combination with a container for holding said illumination device therein, wherein said container comprises an arming pin attached to said container, where upon removal of said illumination device from said container effects removal of said arming pin from between electrical contacts within said illumination device to permit a flow of electricity to said controller.

17. An illumination device according to claim 8, wherein said adhesive is covered by a release member, and said release includes another adhesive on the side of said release member opposite said first adhesive and which is attachable to a surface such that removal of said cradle base from said surface causes said release to remain attached to said surface thereby exposing said first adhesive on said base.

18. A kit, comprising:
a container;
the illumination device of claim 1 within said container; and
at least one cradle within said container, said first cradle shaped to receive and support a portion of said housing therein, said housing being releasably attachable to said cradle.

19. The kit according to claim 18, further comprising a medical instrument such as a retractor or suction device within said container, said instrument having a base portion configured to be removably mountable on said housing and a projecting portion extending from said base portion adjacent to said boom.

20. A kit in accordance with claim 18 further comprising a second cradle shaped to receive and support a portion of said housing therein, said housing being releasably attachable to said second cradle; and
wherein said first cradle includes a mounting strap attached thereto, said strap being further attachable to another item such as a surgical tool so as to be capable of attaching said illumination device to said another item, and said second cradle includes a base having an adhesive thereon for attaching said base to a surface.
